(19) United States
(12) Patent Application Publication
(10) Pub. No.: US 2023/0285599 A1
(43) Pub. Date: Sep. 14, 2023

(54) BIOSENSING COMPOSITION HAVING SENSING GLUCOSE CONTENT, CONTACT LENS AND METHOD OF PREPARATION THEREOF

(71) Applicant: National Yang Ming Chiao Tung University, Hsinchu City (TW)

(72) Inventors: Hsin-Chieh Lin, Hsinchu City (TW); Po-Hsiang Chen, Hsinchu City (TW)

(73) Assignee: National Yang Ming Chiao Tung University, Hsinchu City (TW)

(21) Appl. No.: 18/119,348

(22) Filed: Mar. 9, 2023

Related U.S. Application Data
(60) Provisional application No. 63/269,111, filed on Mar. 10, 2022.

(51) Int. Cl. A61K 49/00; A61B 5/145; A61B 5/00; G02C 7/04

(52) U.S. Cl. CPC A61K 49/0069; A61B 5/14532; A61B 5/6821; G02C 7/04

(57) ABSTRACT

A biosensing composition having sensing glucose content is provided, including a substrate, glucose oxidase, an antioxidant, heme-protein, and a molecular chromogenic chromophore. In addition, a contact lens having sensing glucose content and a method of preparation thereof are provided, so as to achieve the effect of maintaining a low discoloration in normal blood sugar and obvious discoloration in high blood sugar.

### CROSS-REFERENCE TO RELATED APPLICATION

This application claims priority to U.S. Provisional Application Ser. No. 63/269,111, filed on Mar. 10, 2022, which is herein incorporated by reference in its entirety.

### BACKGROUND

### Field of Invention

The present invention relates to a contact lens. More particularly, the present invention relates to a contact lens having sensing glucose content.

### Description of Related Art

Recently, many technologies for detecting glucose in contact lenses mostly use photonic crystals, fluorescent sensing, or electrical signal sensing, but the above technologies are hard in fabrication or sensing. For example, photonic crystals and fluorescence sensors require external devices to detect fluorescence (not easy to carry), and the fluorescence emitted by contact lenses around the eyes may affect the vision of glasses. The technology of sensing glucose using electrical signals has high accuracy, but the technical threshold of the production process is also high, and it takes a long time to test, resulting in high cost.

Therefore, how to provide a vision-friendly, easy-to-carry contact lens that senses glucose content, the related art really needs to be improved.

### SUMMARY

The invention provides a biosensing composition having sensing glucose content, so as to achieve the effect of normal blood sugar maintaining a low discoloration in normal blood sugar and obvious discoloration in high blood sugar.

The present disclosure provides a biosensing composition having sensing glucose content, comprising: a substrate, a glucose oxidase, an antioxidant, a heme-protein, and a molecular chromogenic chromophore.

In some embodiments, the substrate is a polymer formed from monomers.

In some embodiments, before the polymer is polymerized, a material of the polymer comprises 2-hydroxyethylmethacrylate (HEMA), a cross-linking agent, a free radical initiator, or any combination thereof.

In some embodiments, the cross-linking agent is HEMA and ethylene glycol dimethacrylate (EGDMA), the free radical initiator is diphenyl(2,4,6-trimethylbenzoyl)phosphine oxide (TPO), and a weight ratio of HEMA, EGDMA, and TPO are from 100:6:1 to 200:6:1.

In some embodiments, the antioxidant comprises superoxide dismutase, glutathione peroxidase, glutathione reductase, catalase (CAT), glucose-6-phosphodehydrogenase, aldehyde dehydrogenase 3A1, aldehyde dehydrogenase 1A1, uric acid, ascorbic acid, glutathione, L-tyrosine, L-cysteine, nicotinamide adenine dinucleotide phosphate (NADPH), α-tocopherol, retinol, ferritin, albumin, flavonoids, hydroxycinnamates, anthocyanidins, carotenoids, β-carotene, lycopene, 6-hydroxy-2,5,7,8-tetramethylchroman-2-carboxylic acid (Trolox), vitamin C12, kaempferol, or any combination thereof.

In some embodiments, the antioxidant has a weight percentage from 0.0000001% to 20% based on a total weight (100% by weight) of the biosensing composition.

In some embodiments, the heme-protein includes horseradish peroxidase (HRP), porphyrin, hemoglobin, myoglobin, neuroglobin, cytoglobin, leghemoglobin, or any combination thereof.

In some embodiments, the molecular chromogenic chromophore includes 2,2′-azinobis-(3-ethylbenzthiazoline-6-sulphonate)(ABTS), benzidine, benzidine dihydrochloride, N,N′-bis(4-bromophenyl)benzidine), N,N,N′,N′-tetrakis(2-naphthyl)benzidine), N,N′-diphenylbenzidine, N,N,N′,N′-tetramethylbenzidine, 3,3′-diaminobenzidine, 3-amino-9-ethylcarbazole, 3,3′,5,5′-tetramethylbenzidine, 3,3′,5,5′-tetramethylbenzidine dihydrochloride hydrate, 3,3′,5,5′-tetramethylbenzidine dihydrochloride, N-(3-sulfopropyl)-3,3′,5,5′-tetramethylbenzidine sodium salt, cyanine, cyanine salt, 4-methoxy-o-phenylenediamine dihydrochloride, bis(dioxaborine) polymethines, dioxaborine polymethines, β-nicotinamide adenine dinucleotide, β-nicotinamide adenine dinucleotide 2′-phosphate reduced tetrasodium salt hydrate, polymethines, polymethine salt, violaxanthin, 2,2′-azino-bis(3-ethylbenzothiazoline-6-sulfonic acid), 2,2′-azino-bis [3-ethylbenzothiazoline-6-sulfonic acid]-diammonium salt, 3,3′-diaminobenzidine, (1,1′-biphenyl)-3,3′,4,4′-tetramine tetrahydrochloride, 3,3′-diaminobenzidine tetrahydrochloride hydrate, o-dianisidine, o-phenylenediamine dihydrochloride, aniline, polyaniline, polythiophene, poly(3,4-ethylenedioxythiophene), oligoaniline, oligothiophene, oligo(3,4-ethylenedioxythiophene), phenol, thioanisole, 10-Acetyl-3,7-dihydroxyphenoxazine (or also known as Amplex® red), homovanillic acid, or any combination thereof.

In some embodiments, a weight ratio of the glucose oxidase to the antioxidant is from 1:1 to 50:1.

In some embodiments, a weight ratio of the heme-protein to the molecular chromogenic chromophore is from 1:1 to 1:50.

In some embodiments, the biosensing composition having sensing glucose content further comprising an emulsifier.

The present disclosure also provides a contact lens having sensing glucose content, comprising the biosensing composition having sensing glucose content as above mentioned; and a lens body. The biosensing composition having sensing glucose content is disposed on the lens body.

In some embodiments, a substrate of the lens body comprises silicon polymer, poly(2-hydroxyethyl methacrylate) (pHEMA), polyurethane, or any combination thereof.

In some embodiments, a substrate of the lens body comprises HEMA, EGDMA, TPO, methacrylate copolymers (SiMA), 3-[Tris(trimethylsiloxy)silyl]propylmethacrylate (TRIS), or any combination thereof.

In some embodiments, the biosensing composition having sensing glucose content is disposed on the lens body and corresponds to a position of sclera, iris, or pupil.

The present disclosure also provides a method of preparing biosensing composition having sensing glucose content, comprising steps of: mixing and emulsifying a glucose oxidase, an antioxidant, a heme-protein, and a molecular chromogenic chromophore to obtain a mixture; mixing the mixture and a substrate to obtain a substrate mixture; and polymerizing the substrate mixture with light illuminating to obtain the biosensing composition having sensing glucose content.

In some embodiments, the step of mixing and emulsifying comprises: mixing the glucose oxidase, the antioxidant, and an emulsifier to obtain a first mixture; mixing the heme-protein, the molecular chromogenic chromophore and a solvent to obtain a second mixture; and mixing the first mixture and the second mixture to obtain the mixture.

In some embodiments, the emulsifier comprises polypeptide.

In some embodiments, a weight ratio of the glucose oxidase to the antioxidant is from 1:1 to 50:1.

In some embodiments, a weight ratio of the heme-protein to the molecular chromogenic chromophore is from 1:1 to 1:50.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion. The disclosure can be more fully understood by reading the following detailed description of the embodiment, with reference made to the accompanying drawings as follows.

FIG. 1 depicts the light transmittance of biosensing composition having sensing glucose content with or without antioxidant according to some embodiments of the present disclosure.

FIG. 2 depicts the color difference of the three primary colors red (R), green (G) and blue (B) of the biosensing composition having sensing glucose content at different glucose concentrations according to some embodiments of the present disclosure.

FIG. 3 depicts the color contrast of the biosensing composition having sensing glucose content at different glucose concentrations according to some embodiments of the present disclosure.

### DETAILED DESCRIPTION

The following disclosure provides detailed description of many different embodiments, or examples, for implementing different features of the provided subject matter. These are, of course, merely examples and are not intended to limit the invention but to illustrate it. In addition, various embodiments disclosed below may combine or substitute one embodiment with another, and may have additional embodiments in addition to those described below in a beneficial way without further description or explanation. In the following description, many specific details are set forth to provide a more thorough understanding of the present disclosure. It will be apparent, however, to those skilled in the art, that the present disclosure may be practiced without these specific details.

Further, spatially relative terms, such as “beneath,” “over” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, or “includes” and/or “including” or “has” and/or “having” when used in this specification, specify the presence of stated features, regions, integers, steps, operations, elements, and/or components, but do not preclude the presence or addition of one or more other features, regions, integers, steps, operations, elements, components, and/or groups thereof.

In some embodiments of the present disclosure, the contact lens sensing glucose has warning effect to who has diabetes during high blood sugar under actual use. The present disclosure does not need to use cumbersome comparison methods such as photodegradable nanoparticles or color cards. It only needs to use antioxidant to achieve a high contrast glucose content of contact lens that maintains a low discoloration state for a long time in normal blood sugar and obvious discoloration in high blood sugar. For example, the present disclosure can be easily analyzed by mirror reflection, mobile phone imaging method, or mobile phone application (APP). When a significant color change in a specific area (including biosensing composition having sensing glucose content) of the contact lens is observed (for example, when blood sugar reaches to 1.5 mM or more), user can quickly determine whether you have hyperglycemia, and then perform follow-up medical treatment immediately.

As used herein, “biosensing composition” can also refer to biosensing material or biosensor.

In some embodiments, the biosensing composition having sensing glucose content includes substrate, glucose oxidase, antioxidant, heme-protein, and molecular chromogenic chromophore.

In some embodiments, in the biosensing composition having sensing glucose content, a weight percentage of the antioxidant is from 0.0000001% to 20% based on the total weight (100% by weight) of the biosensing composition, such as 0.0000002%, 0.000002%, 0.00002%, 0.0002%, 0.002%, 0.02%, 0.04%, 0.06%, 0.08%, 0.1%, 0.2%, 0.3%, 0.4%, 0.5%, 0.6%, 0.7%, 0.8%, 0.9%, 1.0%, 1.5%, 2.0%, 3.0%, 4.0%, 5.0%, 6.0%, 7.0%, 8.0%, 9.0%, 10%, 11%, 12%, 13%, 14%, 15%, 16%, 17%, 18%, 19%, or any value between any two of these values.

In some embodiments, a weight ratio of the glucose oxidase to the antioxidant is from 1:1 to 50:1, such as 2:1, 3:1, 4:1, 5:1, 6:1, 7:1, 8:1, 9:1, 10:1, 15:1, 20:1, 25:1, 30:1, 35:1, 40:1, 45:1, or any value between any two of these values.

In some embodiments, a weight ratio of the heme-protein to molecular chromogenic chromophore is from 1:10 to 1:50, such as 1:15, 1:20, 1:25, 1:30, 1:35, 1:40, 1:45, or any value between any two of these values.

In some embodiments, the biosensing composition having sensing glucose content further comprises an emulsifier, in which the emulsifier can be added with a first mixture comprising the glucose oxidase and the antioxidant at the same time, can also be added with a second mixture comprising the heme-protein and the molecular chromogenic chromophore at the same time, or can also be added when mixing the glucose oxidase, the antioxidant, the heme-protein, and the molecular chromogenic chromophore at the same time during the preparation of the biosensing composition, so as to facilitate the biosensing composition be evenly emulsified.

In some embodiments, biosensing composition having sensing glucose content further comprises a cross-linking agent for stable combination of the glucose oxidase and the antioxidant. In some examples, the cross-linking agent includes, but is not limited to geniposide, also called genipin.

In some embodiments, the biosensing composition or the biosensing contact lens having sensing glucose content, the steps of polymerizing the substrate mixture with light illuminating is to illuminate the substrate mixture with ultraviolet light for a period of time until the polymerization is completed. In some embodiments, the period of time is about 5 minutes to 45 minutes, such as 10 minutes, 15 minutes, 20 minutes, 25 minutes, 30 minutes, 35 minutes, 40 minutes, or any value between any two of these values.

In some embodiments, the lens body may include any suitable polymeric material known in the art. For example, the lens body may be a silicone lens body or non-silicone lens body without siloxane component. More specifically, the lens body may include polymeric material such as silicone hydrogel, poly(2-hydroxyethyl methacrylate) (pHEMA), or polyurethane. For example, the lens body may include senofilcon A, falcon V, balafilcon A, somofilcon A, etafilcon A, hilafilcon B, ocufilcon D, or nelfilcon A.

In some embodiments, for the production of contact lenses, the biosensing composition is cut into the desired shape and then attached to the cavity mold. In some other embodiments, the cut shape of the biosensing composition includes, but is not limited to circle, square, triangle, polygon, etc.

In some embodiments, the biosensing composition covers the middle layer, a front surface, a rear surface, or any combination thereof of the lens body.

In some embodiments, the biosensing composition having sensing glucose content is a discoloration area that is a polymer film including glucose oxidase (GOx), HRP, ABTS, HEMA and the antioxidant. The lens body of the contact lens is placed in the mold before the lens body is polymerized into a solid state. After being polymerized by ultraviolet light, the discoloration area is embedded in the lens body.

In some embodiments, glucose in tears diffuses from the outside of the lens to the discolored area through diffusion, so that GOx in the discolored area reacts with glucose to produce hydrogen peroxide. Hydrogen peroxide reacts with ABTS, and the original colorless and transparent ABTS is oxidized to green ABTS⁺ under the catalysis of HRP.

A number of examples are provided herein to elaborate the biosensing composition having sensing glucose content of the instant disclosure. However, the examples are for demonstration purpose alone, and the instant disclosure is not limited thereto.

### Example

Although a series of operations or steps are used below to describe the method disclosed herein, an order of these operations or steps should not be construed as a limitation to the present invention. For example, some operations or steps may be performed in a different order and/or other steps may be performed at the same time. In addition, all shown operations, steps and/or features are not required to be executed to implement an embodiment of the present invention. In addition, each operation or step described herein may include a plurality of sub-steps or actions.

For the sake of clarity, features and elements that are known in the art and are not essential to an understanding of the principles described will be omitted.

### Example 1

96.66 wt %:3.34 wt % (28:1) of GOx/antioxidant CAT were prepared and polypeptide were added to fully dissolve and emulsify to obtain a first mixture. Next, 4.58 wt %:95.42 wt % (1:20) of HRP/ABTS with phosphate buffered saline (PBS) were mixed to obtain a second mixture. Next, the first mixture and the second mixture were mixed to obtain a mixture. Next, 96.62 wt %:2.90 wt %:0.48 wt % (200:6:1) of HEMA:EGDMA:TPO as a substrate of a biosensing composition were prepared. Next, the mixture and the substrate were mixed and fully stirred to obtain a substrate mixture. Next, the substrate mixture was slowly dropped on the glass mold and polymerized under 365 nanometer (nm) ultraviolet light, and then the glass mold was removed and demolded with deionized water to obtain the biosensing composition. In the aspect of contact lens production, the biosensing composition was cut into circles and then attached to the cavity mold (female mold), and then 96.62 wt %:2.90 wt %:0.48 wt % (200:6:1) of HEMA:EGDMA:TPO substrate as the material of the contact lens were added into the cavity mold. Next, the core mold (male mold) was covered and the biosensing composition and the substrate were polymerized under 365 nm ultraviolet light. After the polymerization was completed, the mold was demolded to obtain a contact lens having sensing glucose content.

### Example 2

93.33 wt %:6.67 wt % (14:1) of GOx/antioxidant CAT were prepared and polypeptide were added to fully dissolve and emulsify to obtain a first mixture. Next, 4.58 wt %:95.42 wt % (1:20) of HRP/ABTS with PBS were mixed to obtain a second mixture. Next, the first mixture and the second mixture were mixed to obtain a mixture. Next, 96.62 wt %:2.90 wt %:0.48 wt % (200:6:1) of HEMA:EGDMA:TPO as a substrate of a biosensing composition were prepared. Next, the mixture and the substrate were mixed and fully stirred to obtain a substrate mixture. Next, the substrate mixture was slowly dropped on the glass mold and polymerized under 365 nm ultraviolet light, and then the glass mold was removed and demolded with deionized water to obtain the biosensing composition. In the aspect of contact lens production, the biosensing composition was cut into circles and then attached to the cavity mold (female mold), and then 96.62 wt %:2.90 wt %:0.48 wt % (200:6:1) of HEMA:EGDMA:TPO substrate as the material of the contact lens were added into the cavity mold. Next, the core mold (male mold) was covered and the biosensing composition and the substrate were polymerized under 365 nm ultraviolet light. After the polymerization was completed, the mold was demolded to obtain a contact lens having sensing glucose content.

### Example 3

54.55 wt %:45.45 wt % (5:4) of GOx/antioxidant sodium ascorbate were prepared and polypeptide were added to fully dissolve and emulsify to obtain a first mixture. Next, 4.58 wt %:95.42 wt % (1:20) of HRP/ABTS with PBS were mixed to obtain a second mixture. Next, the first mixture and the second mixture were mixed to obtain a mixture. Next, 96.62 wt %:2.90 wt %:0.48 wt % (200:6:1) of HEMA:EGDMA:TPO as a substrate of a biosensing composition were prepared. Next, the mixture and the substrate were mixed and fully stirred to obtain a substrate mixture. Next, the substrate mixture was slowly dropped on the glass mold and polymerized under 365 nm ultraviolet light, and then the glass mold was removed and demolded with deionized water to obtain the biosensing composition. In the aspect of contact lens production, the biosensing composition was cut into circles and then attached to the cavity mold (female mold), and then 96.62 wt %:2.90 wt %:0.48 wt % (200:6:1) of HEMA:EGDMA:TPO substrate as the material of the contact lens were added into the cavity mold. Next, the core mold (male mold) was covered and the biosensing composition and the substrate were polymerized under 365 nm ultraviolet light. After the polymerization was completed, the mold was demolded to obtain a contact lens having sensing glucose content.

### Example 4

18.63 wt %:3.73 wt %:77.64 wt % (5:1:20) of GOx:HRP:ABTS and saturated Trolox (˜3 mg/mL) were prepared and polypeptide were added to fully dissolve and emulsify to obtain a mixture. Next, 96.62 wt %:2.90 wt %:0.48 wt % (200:6:1) of HEMA:EGDMA:TPO as a substrate of a biosensing composition were prepared. Next, the mixture and the substrate were mixed and fully stirred to obtain a substrate mixture. Next, the substrate mixture was slowly dropped on the glass mold and polymerized under 365 nm ultraviolet light, and then the glass mold was removed and demolded with deionized water to obtain the biosensing composition. In the aspect of contact lens production, the biosensing composition was cut into circles and then attached to the cavity mold (female mold), and then 96.62 wt %:2.90 wt %:0.48 wt % (200:6:1) of HEMA:EGDMA:TPO substrate as the material of the contact lens were added into the cavity mold. Next, the core mold (male mold) was covered and the biosensing composition and the substrate were polymerized under 365 nm ultraviolet light. After the polymerization was completed, the mold was demolded to obtain a contact lens having sensing glucose content.

### Example 5

8.33 wt %:8.33 wt %:83.34 wt % (1:1:10) of GOx:CAT:genipin were dissolved in PBS, then polypeptide were added to fully dissolve and emulsify, and then excessed genipin was removed by centrifugation to obtain a first mixture. Next, 9.09 wt %:90.91 wt % (1:10) of HRP/ABTS were dissolved in the first mixture to obtain a mixture. Next, 96.62 wt %:2.90 wt %:0.48 wt % (200:6:1) of HEMA:EGDMA:TPO as a substrate of a biosensing composition were prepared. Next, the mixture and the substrate were mixed and fully stirred to obtain a substrate mixture. Next, the substrate mixture was slowly dropped on the glass mold and polymerized under 365 nm ultraviolet light, and then the glass mold was removed and demolded with deionized water to obtain the biosensing composition. In the aspect of contact lens production, the biosensing composition was cut into circles and then attached to the cavity mold (female mold), and then 96.62 wt %:2.90 wt %:0.48 wt % (200:6:1) of HEMA:EGDMA:TPO substrate as the material of the contact lens were added into the cavity mold. Next, the core mold (male mold) was covered and the biosensing composition and the substrate were polymerized under 365 nm ultraviolet light. After the polymerization was completed, the mold was demolded to obtain a contact lens having sensing glucose content.

### Example 6

96.66 wt %:3.34 wt % (28:1) of GOx/antioxidant CAT were prepared and polypeptide were added to fully dissolve and emulsify to obtain a first mixture. Next, 4.58 wt %:95.42 wt % (1:20) of HRP/ABTS with PBS were mixed to obtain a second mixture. Next, the first mixture and the second mixture were mixed to obtain a mixture. Next, 96.62 wt %:2.90 wt %:0.48 wt % (200:6:1) of HEMA:EGDMA:TPO as a substrate of a biosensing composition were prepared. Next, the mixture and the substrate were mixed and fully stirred to obtain a substrate mixture. Next, the substrate mixture was slowly dropped on the glass mold and polymerized under 365 nm ultraviolet light, and then the glass mold was removed and demolded with deionized water to obtain the biosensing composition. In the aspect of contact lens production, the biosensing composition was cut into circles and then attached to the cavity mold (female mold), and then 72.40 wt %:18.10 wt %:9.05 wt %:0.45 wt % (160:40:20:1) of HEMA:SiMA:TRIS:TPO substrate as the material of the contact lens were added into the cavity mold. Next, the core mold (male mold) was covered and the biosensing composition and the substrate were polymerized under 365 nm ultraviolet light. After the polymerization was completed, the mold was demolded to obtain a contact lens having sensing glucose content.

### Comparative Example 1

The preparation method of comparative example 1 is similar to example 1, the difference is that comparative example 1 did not add antioxidant. Specifically, Gox added in to polypeptide were fully dissolved and emulsified to obtain a first mixture. Next, 4.58 wt %:95.42 wt % (1:20) of HRP/ABTS with PBS were mixed to obtain a second mixture. Next, the first mixture and the second mixture were mixed to obtain a mixture. Next, 96.62 wt %:2.90 wt %:0.48 wt % (200:6:1) of HEMA:EGDMA:TPO as a substrate of a biosensing composition were prepared. Next, the mixture and the substrate were mixed and fully stirred to obtain a substrate mixture. Next, the substrate mixture was slowly dropped on the glass mold and polymerized under 365 nm ultraviolet light, and then the glass mold was removed and demolded with deionized water to obtain the biosensing composition. In the aspect of contact lens production, the biosensing composition was cut into circles and then attached to the cavity mold (female mold), and then 96.62 wt %:2.90 wt %:0.48 wt % (200:6:1) of HEMA:EGDMA:TPO substrate as the material of the contact lens were added into the cavity mold. Next, the core mold (male mold) was covered and the biosensing composition and the substrate were polymerized under 365 nm ultraviolet light. After the polymerization was completed, the mold was demolded to obtain a contact lens having sensing glucose content.

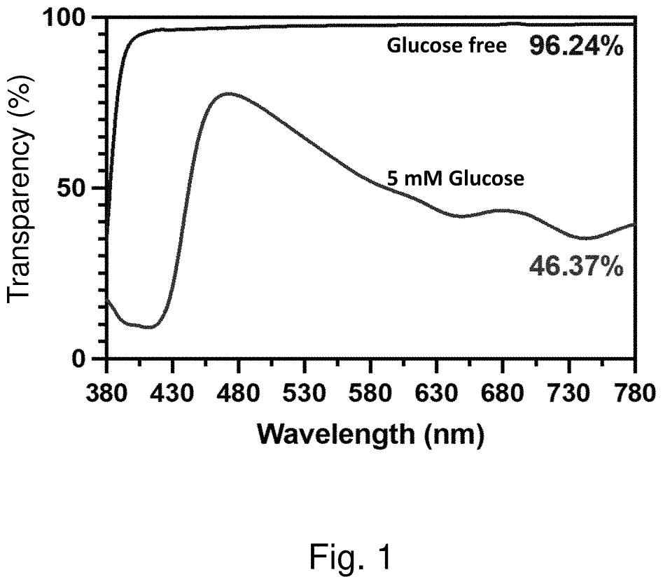

Please refer to FIG. 1, FIG. 1 depicts the light transmittance of biosensing composition having sensing glucose content with or without antioxidant according to some embodiments of the present disclosure. Since the molecular chromogenic chromophore of the biosensing composition was ABTS, the originally colorless and transparent ABTS was oxidized to green ABTS⁺ when encountering glucose, in which the wavelength of visible light was from 380 nm to 780 nm. Example 2 was tested before and after soaking in 5 mM glucose solution. FIG. 1 shows that the biosensing composition exhibited high light transmittance at all wavelengths before the biosensing composition immersing in glucose solution. When the biosensing composition immersed in a 5 mM glucose solution, only in the green wavelength range (495 nm to 570 nm) had a high penetration (greater than 50%) and appearing in green. Thus, it can be used as an indicator of changes in glucose concentration.

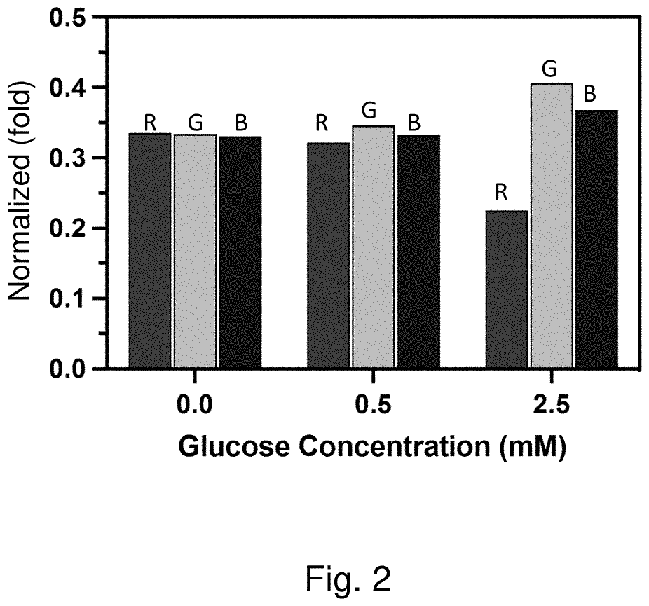

Please refer to FIG. 2, FIG. 2 depicts the color difference of the three primary colors red (R), green (G) and blue (B) of the biosensing composition having sensing glucose content at different glucose concentrations according to some embodiments of the present disclosure. Samples of Example 2 respectively immersed in 0 mM, 0.5 mM, and 2.5 mM glucose solution were tested, and the three primary colors were standardized with the calculation method as follows formula I to formula III:

$$Normalized R = \frac{R}{R + G + B} , \qquad formula I$$

$$Normalized G = \frac{G}{R + G + B} , \qquad formula II$$

$$Normalized B = \frac{B}{R + G + B} . \qquad formula III$$

FIG. 2 shows the ratio distribution of the three primary colors red (R) green (G) and blue (B). It can be observed that when the ratio of green (G) was from 0 to 0.5, there was no significant difference. However, it was surprisingly found that as the glucose concentration increased to 2.5 mM, the proportion of green (G) increased significantly while the proportion of the complementary color red (R) decreased significantly.

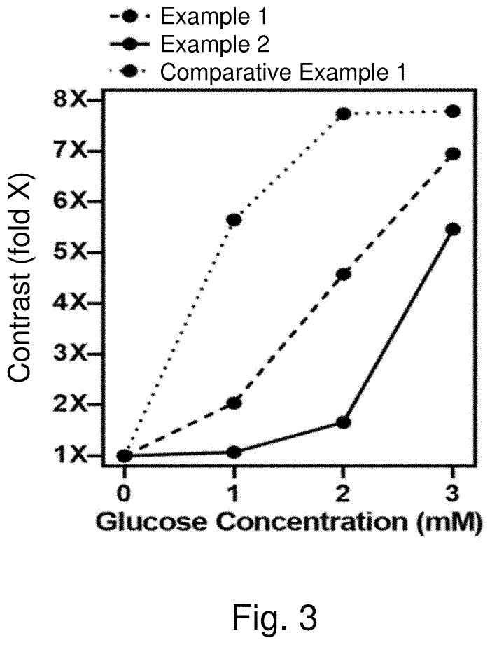

Please refer to FIG. 3, FIG. 3 depicts the color contrast of the biosensing composition having sensing glucose content at different glucose concentrations according to some embodiments of the present disclosure. The dash line is example 1, the solid line is example 2, and the dot line is comparative example 1. The determination of contrast was based on the color difference between each example and the standard light source D65 white light (color temperature was 6500K), or between comparative example and the standard light source D65 white light under different glucose concentrations.

As shown in FIG. 3, when the glucose concentration is low (for example, 1 mM), the contrast of Comparative Example 1 lacking the antioxidant increased up to 6 times obviously. On the contrary, example 1 with antioxidant still maintained below 2 times, and even example 2 almost maintained no color change. In other words, comparative example 1 lacking antioxidant could not realize the effect of no discoloration at low glucose concentration (less than 1.5 mM) and could not obvious discolor at high concentration (greater than 1.5 mM). That is, when the biosensing composition lacks the antioxidant, the discoloration effect cannot be controlled. Other examples 3 to 6 also had the effect of a low discoloration at low glucose concentration and obvious discoloration at high glucose concentration (figure not shown).

In some embodiments, the contact lens having sensing glucose content of the present disclosure has discolored response and can return to transparent after repeating about 30 to 50 times, which can still effectively respond the changes in blood sugar concentration.

The present disclosure provides a biosensing composition having sensing glucose content, so as to achieve the effect of normal blood sugar maintaining a low discoloration in normal blood sugar and obvious discoloration in high blood sugar.

While the disclosure has been described by way of example(s) and in terms of the preferred embodiment(s), it is to be understood that the disclosure is not limited thereto. On the contrary, it is intended to cover various modifications and similar arrangements and procedures, and the scope of the appended claims therefore should be accorded the broadest interpretation so as to encompass all such modifications and similar arrangements and procedures.

1. A biosensing composition having sensing glucose content, comprising: a substrate, a glucose oxidase, an antioxidant, a heme-protein, and a molecular chromogenic chromophore.

2. The biosensing composition having sensing glucose content of claim 1, wherein the substrate is a polymer formed from monomers.

3. The biosensing composition having sensing glucose content of claim 2, wherein before the polymer is polymerized, a material of the polymer comprises 2-hydroxyethylmethacrylate (HEMA), a cross-linking agent, a free radical initiator, or any combination thereof.

4. The biosensing composition having sensing glucose content of claim 3, wherein the cross-linking agent is HEMA and ethylene glycol dimethacrylate (EGDMA), the free radical initiator is diphenyl(2,4,6-trimethylbenzoyl)phosphine oxide (TPO), and a weight ratio of HEMA, EGDMA, and TPO are from 100:6:1 to 200:6:1.

5. The biosensing composition having sensing glucose content of claim 1, wherein the antioxidant comprises superoxide dismutase, glutathione peroxidase, glutathione reductase, catalase (CAT), glucose-6-phosphodehydrogenase, aldehyde dehydrogenase 3A1, aldehyde dehydrogenase 1A1, uric acid, ascorbic acid, glutathione, L-tyrosine, L-cysteine, nicotinamide adenine dinucleotide phosphate (NADPH), α-tocopherol, retinol, ferritin, albumin, flavonoids, hydroxycinnamates, anthocyanidins, carotenoids, β-carotene, lycopene, 6-hydroxy-2,5,7,8-tetramethylchroman-2-carboxylic acid (Trolox), vitamin C12, kaempferol, or any combination thereof.

6. The biosensing composition having sensing glucose content of claim 1, wherein the antioxidant has a weight percentage from 0.0000001% to 20% based on a total weight (100% by weight) of the biosensing composition.

7. The biosensing composition having sensing glucose content of claim 1, wherein the heme-protein includes horseradish peroxidase (HRP), porphyrin, hemoglobin, myoglobin, neuroglobin, cytoglobin, leghemoglobin, or any combination thereof.

8. The biosensing composition having sensing glucose content of claim 1, wherein the molecular chromogenic chromophore includes 2,2′-azinobis-(3-ethylbenzthiazoline-6-sulphonate)(ABTS), benzidine, benzidine dihydrochloride, N,N′-bis(4-bromophenyl)benzidine), N,N,N′,N′-tetrakis(2-naphthyl)benzidine), N,N′-diphenylbenzidine, N,N,N′,N′-tetramethylbenzidine, 3,3′-diaminobenzidine, 3-amino-9-ethylcarbazole, 3,3′,5,5′-tetramethylbenzidine, 3,3′,5,5′-tetramethylbenzidine dihydrochloride hydrate, 3,3′,5,5′-tetramethylbenzidine dihydrochloride, N-(3-sulfopropyl)-3,3′,5,5′-tetramethylbenzidine sodium salt, cyanine, cyanine salt, 4-methoxy-o-phenylenediamine dihydrochloride, bis(dioxaborine) polymethines, dioxaborine polymethines, β-nicotinamide adenine dinucleotide, β-nicotinamide adenine dinucleotide 2′-phosphate reduced tetrasodium salt hydrate, polymethines, polymethine salt, violaxanthin, 2,2′-azino-bis(3-ethylbenzothiazoline-6-sulfonic acid), 2,2′-azino-bis [3-ethylbenzothiazoline-6-sulfonic acid]-diammonium salt, 3,3′-diaminobenzidine, (1,1′-biphenyl)-3,3′,4,4′-tetramine tetrahydrochloride, 3,3′-diaminobenzidine tetrahydrochloride hydrate, o-dianisidine, o-phenylenediamine dihydrochloride, aniline, polyaniline, polythiophene, poly(3,4-ethylenedioxythiophene), oligoaniline, oligothiophene, oligo(3,4-ethylenedioxythiophene), phenol, thioanisole, 10-Acetyl-3,7-dihydroxyphenoxazine, homovanillic acid, or any combination thereof.

9. The biosensing composition having sensing glucose content of claim 1, wherein a weight ratio of the glucose oxidase to the antioxidant is from 1:1 to 50:1.

10. The biosensing composition having sensing glucose content of claim 1, wherein a weight ratio of the heme-protein to the molecular chromogenic chromophore is from 1:1 to 1:50.

11. The biosensing composition having sensing glucose content of claim 1, further comprising an emulsifier.

12. A contact lens having sensing glucose content, comprising:
the biosensing composition having sensing glucose content of claim 1; and
a lens body, the biosensing composition having sensing glucose content disposed on the lens body.

13. The contact lens having sensing glucose content of claim 12, wherein a substrate of the lens body comprises silicon polymer, poly(2-hydroxyethyl methacrylate) (pHEMA), polyurethane, or any combination thereof.

14. The contact lens having sensing glucose content of claim 12, wherein a substrate of the lens body comprises HEMA, EGDMA, TPO, methacrylate copolymers (SiMA), 3-[Tris(trimethylsiloxy)silyl]propylmethacrylate (TRIS), or any combination thereof.

15. The contact lens having sensing glucose content of claim 12, wherein the biosensing composition having sensing glucose content is disposed on the lens body and corresponds to a position of sclera, iris, or pupil.

16. A method of preparing biosensing composition having sensing glucose content, comprising steps of:
mixing and emulsifying a glucose oxidase, an antioxidant, a heme-protein, and a molecular chromogenic chromophore to obtain a mixture;
mixing the mixture and a substrate to obtain a substrate mixture; and
polymerizing the substrate mixture with light illuminating to obtain the biosensing composition having sensing glucose content.

17. The method of claim 16, wherein the step of mixing and emulsifying comprises:
mixing the glucose oxidase, the antioxidant, and an emulsifier to obtain a first mixture;
mixing the heme-protein, the molecular chromogenic chromophore and a solvent to obtain a second mixture; and
mixing the first mixture and the second mixture to obtain the mixture.

18. The method of claim 17, wherein the emulsifier comprises polypeptide.

19. The method of claim 16, wherein a weight ratio of the glucose oxidase to the antioxidant is from 1:1 to 50:1.

20. The method of claim 16, wherein a weight ratio of the heme-protein to the molecular chromogenic chromophore is from 1:1 to 1:50.
